(19) United States
(12) Patent Application Publication
(10) Pub. No.: US 2026/0034079 A1
(43) Pub. Date: Feb. 5, 2026

(54) PHARMACEUTICAL COMPOSITION CONTAINING HYDROXYUREA FOR USE IN THE TREATMENT OF SEVERE CHRONIC ANEMIA

(71) Applicant: THERAVIA, NEUILLY-SUR-SEINE (FR)

(72) Inventors: Corinne DUGUET, PARIS (FR); Isabelle DUPUIS, NEUILLY (FR)

(21) Appl. No.: 19/358,252

(22) Filed: Oct. 14, 2025

Related U.S. Application Data
(63) Continuation of application No. 17/433,869.

(30) Foreign Application Priority Data
Feb. 28, 2019 (FR) 1902093

(51) Int. Cl. A61K 31/17; A61P 7/06

(52) U.S. Cl. CPC A61K 31/17; A61P 7/06

(57) ABSTRACT

The invention relates to the method of use of a pharmaceutical composition containing hydroxyurea, for the treatment of severe chronic anemia in a patient suffering from sickle cell anemia not already undergoing treatment with hydroxyurea and not suffering a vaso-occlusive crisis.

The present invention relates to a pharmaceutical composition comprising hydroxyurea, in the scope of the treatment of severe chronic anaemia in a patient suffering from sickle cell disease, and not already undergoing treatment with hydroxyurea or not suffering vaso-occlusive crisis. Sickle cell disease is a genetic disease resulting from an abnormality of haemoglobin, a protein in red blood cells that carries oxygen in the blood. It is the most common genetic disease in France, and probably in the world, with an estimate of more than 100 million people affected. Approximately 80% of the sickle cell disease cases are concentrated in sub-Saharan Africa. The disease is also quite common in parts of India, the Arabian Peninsula and among the populations of African descent scattered around the world.

Sickle cell disease is caused by the substitution of a single amino acid residue on the β-chain of haemoglobin. This genetic mutation-replacement of the glutamate in position 6 by a valine. resulting in haemoglobin S-favors, under certain conditions, the appearance of rigid red blood cells with an elongated shape, known as a sickle or acanthus leaf shape. In addition to being deformed, the sickle-shaped red blood cells are more fragile and rigid than the normal red blood cells. They circulate poorly in the vessels, which prevents them from fully playing their role as oxygen carriers. In particular, they hemolyze easily in fine capillaries.

The three main manifestations of sickle cell disease are the anaemia, the vaso-occlusive crisis and a reduced resistance to certain infections, which can manifest themselves in very different ways depending on the cases.

The anaemia refers to a lack of haemoglobin and results in an excessive fatigue and a feeling of weakness. The red blood cells, which are constantly renewed, are produced in the center of the bones, in the red bone marrow. From there, they pass into the general flow where they normally remain for 120 days in the blood flow and are then destroyed in the spleen. In case of sickle cell disease, the red blood cells shaped like a sickle are more fragile and are considered abnormal by the spleen. They are thus rapidly destroyed, which causes anaemia in particular a severe chronic anaemia, i.e. in particular an anaemia characterized in that the total haemoglobin level is less than or equal to 7.5 g/dl of blood.

Other cells are involved m the pathophysiology of the vaso-occlusive crisis: the endothelial cells, the reticulocytes, the neutrophils, the blood platelets. However, the mononuclear cells and the platelets have an abnormal composition of polyunsaturated fatty acids in the sickle cell patients.

These crisis can be very painful. These pains are the most frequent manifestations of the disease, they can be sudden and transitory or chronic. They can be triggered by a change in temperature, by stress, by the dehydration, as well as by a high altitude.

The infections are one of the most common complications of sickle cell disease. They can occur throughout the life of the sickle cell patient and can be life-threatening, especially in infants and young children. The bacterial infection is susceptible to rapid spread and serious localization such as meningitis or osteomyelitis. The pneumococcus and the salmonella are the most common bacteria.

To date, we do not know how to cure tsickle cell disease, it is simply possible to relieve the pain in times of crisis and to prevent the serious infections as much as possible. The management of sickle cell disease consists in particular of preventing the infections with vaccines and antibiotics, ensuring a proper hydration of the body, treating the pain caused by the crisis, and even a vitamin By (folic acid) supplementation. A blood transfusion or a hydroxyurea (hydroxycarbamide) administration may also be required in case of painful crisis A bone marrow transplant can lead to a cure in a small number of cases.

However, to date, there is no treatment that allows a sustained improvement of the severe chronic anaemia in the patients with sickle cell disease who are not already undergoing treatment with hydroxyurea or who do not have vaso-occlusive crisis.

Thus, according to a first aspect, the invention relates to a pharmaceutical composition comprising hydroxyurea as active substance, for use in the treatment of severe chronic anaemia in a patient suffering from sickle cell disease and not already undergoing treatment with hydroxyurea, the total haemoglobin level in this patient being, before said use of hydroxyurea, less than or equal to 7.5 g/dl of blood.

The invention also relates to a pharmaceutical composition comprising hydroxyurea as an active substance, for use in the treatment of severe chronic anaemia in a patient suffering from sickle cell disease, and not having vaso-occlusive crisis, the total haemoglobin level in this patient being, before said use of hydroxyurea, less than or equal to 7.5 g/dl of blood. Surprisingly, the inventors have demonstrated that hydroxyurea can be used to effectively treat the severe chronic anaemia in the patients with sickle cell disease, and not already undergoing treatment with hydroxyurea. In particular, the hydroxyurea can be used to effectively treat severe to very severe chronic anaemia in the patients with sickle cell disease who do not have vaso-occlusive crisis.

It should be noted that hydroxyurea is known, in particular at high doses, to have myelosuppressive effects and/or to lower the total haemoglobin level, which is contrary to a treatment of anaemia (i.e. a lack of haemoglobin). The invention also relates to a pharmaceutical composition for use in the treatment of severe chronic anaemia in a patient suffering from sickle cell disease, and not already undergoing treatment with hydroxyurea, the total haemoglobin level in this patient being, before use of hydroxyurea, less than 7.5 g/dl of blood, to increase the total haemoglobin level in this patient.

The invention also relates to a pharmaceutical composition for use in the treatment of severe chronic anaemia in a patient suffering from sickle cell disease, and not having vaso-occlusive crisis, the total haemoglobin level in this patient being, before use of hydroxyurea, less than 7.5 g/dl of blood, to increase the total haemoglobin level in this patient.

According to an embodiment, the total haemoglobin level in the patient, before use of hydroxyurea is less than 7.4; 7.3; 7.2; 7.1; 7.0; 6.9; 6.8; 6.7; 6.6; 6.5; 6.4; 6.3; 6.2; 6.1 or 6.0 g/dl of blood.

According to an embodiment, the total haemoglobin level in the patient, before use of hydroxyurea, is greater than 5.0 g/dl of blood, in particular greater than 5.1; 5.2; 5.3; 5.4; 5.5; 5.6; 5.7; 5.8; 5.9 or 6.0 g/dl of blood.

According to an embodiment, the total haemoglobin level in the patient, before use of hydroxyurea, is greater than 5.0 g/dl of blood, in particular greater than 5.1; 5.2; 5.3; 5.4; 5.5; 5.6; 5.7; 5.8; 5.9; 6.0; 6.1; 6.2; 6.3; 6.4 or 6.5 g/dl of blood. According to an embodiment, the total haemoglobin level in the patient, before use of hydroxyurea, as less than 7.5:7.4; 7.3; 7.2; 7.1; 7.0; 6.9; 6.8; 6.7; 6.6; 6.5; 6.4; 6.3; 6.2 or 6.1 g/dl of blood, and greater than 5.0; 5.1; 5.2; 5.3; 5.4; 5.5; 5.6; 5.7; 5.8 or 5.9 g/dl of blood.

According to an embodiment, the total haemoglobin level in the patient, before use of hydroxyurea is less than 7.5; 7.4; 7.3; 7.2; 7.1; 7.0; 6.9; 6.8; 6.7; 6.6 or 6.5 g/dl of blood, and greater than 5.0; 5.1; 5.2; 5.3; 5.4; 5.5; 5.6; 5.7; 5.8; 5.9; 6.0; 6.1; 6.2; 6.3 or 6.4 g/dl of blood.

For example, the total haemoglobin level in the patient, before use of hydroxyurea, is from 6.0 to 7.5 g/dl of blood, or from 6.5 to 7.5 g/dl of blood, or from 6.0 to 7.0 g/dl of blood, or from 6.0 to 6.5 g/dl of blood, or from 7.0 to 7.5 g/dl of blood, or from 6.5 to 7.0 g/dl of blood.

According to an embodiment, the fetal haemoglobin level varies between 5 and 25% of the total haemoglobin level in the patient, before hydroxyurea use, in particular between 10 and 25% of the total haemoglobin level in the patient.

According to an embodiment, the patient is aged, before use of hydroxyurea, of 9 months or more, in particular of 2 years or more, the patient being in particular an adult, or aged of 1 to 18 years old, in particular of 3 to 13 years old.

According to an embodiment, the total haemoglobin level in the patient increases by more than 5%, in particular more than 6, 7, 8, 9 or 10%, in particular after 6 to 18 months of hydroxyurea use, more particularly by more than 8%, e. g. 9, 10, 11, 12, 13, 14 or 15%, in particular after 12 to 24 months of hydroxyurea use.

According to an embodiment, the total haemoglobin level in the patient increases to more than 7.7 g/dl of blood, in particular after 6 to 18 months of hydroxyurea use.

According to an embodiment, the total haemoglobin level in the patient increases to more than 8.0 g/dl of blood, in particular after 12 to 24 months of hydroxyurea use.

According to an embodiment, the pharmaceutical composition comprises hydroxyurea in an included amount of 50 to 1000 mg.

According to an embodiment, the pharmaceutical composition is administered at a dose of 10 to 40 mg/kg/day, in particular of 10 to 35 mg/kg/day, more particularly of 15 to 30 mg/kg/day. According to an embodiment, the pharmaceutical composition comprises at least one pharmaceutically acceptable carrier.

According to an embodiment, the pharmaceutical composition is a tablet, in particular a rapidly disintegrating tablet or a dispersible film-coated tablet According to an embodiment, the pharmaceutical composition is a liquid pharmaceutical form, for example a syrup or a drinkable solution.

According to an embodiment, the tablet further comprises one or more additional ingredients well known to the person skilled in the art, such as, in particular, the diluents, the binders, the lubricants, the coating agents and optionally the sweeteners.

According to an advantageous embodiment, these additional ingredients are present in the tablet in an amount of 5 to 85% by weight, in particular 10 to 70%, more particularly 20 to 60%, with respect to the total weight of the uncoated tablet, the remainder of the tablet consisting in particular of hydroxyurea.

The diluent is in particular selected from the group comprising the polyols with less than 13 carbon atoms, in particular mannitol, xylitol, sorbitol, maltitol, lactitol, microcrystalline celluloses, starch (in particular corn, rice, potato), pregelatinized starch, sugars and derivatives, in particular maltose, fructose, sucrose, dicalcium phosphate and its derivatives, glycine and other pharmaceutically compatible amino acids, and their derivatives, lactose and its derivatives, dextrin and its derivatives, calcium carbonate, calcium lactate, calcium phosphate, calcium sulfate, silica, and their mixtures.

According to an advantageous embodiment, the diluent is a microcrystalline cellulose or one of its derivatives, in particular in a mixture with silica (silicified microcrystalline cellulose).

According to an advantageous embodiment, the diluent is present in the tablet in an amount of 5 to 85% by weight, in particular 10 to 70%, more particularly 20 to 60%, with respect to the total weight of the uncoated tablet.

According to an advantageous embodiment, the diluent is also a binder.

If this is not the case, the binder is in particular chosen from the group comprising alginic acid, alginates, in particular sodium alginate, starch, pregelatinized starch, carboxymethyl cellulose, dextrins, gelatin, glucose syrup, guar gum, hydrogenated vegetable oils, carbomers, methylcellulose, ethyl cellulose, hydroxyethyl methylcellulose, hydroxyethyl cellulose, hydroxypropyl cellulose, hydroxypropyl methylcellulose, dextrins, hypromellose, poly dextrose, magnesium and aluminum silicate, maltodextrins, methylcellulose, polyethylene oxide, polymethacrylates, povidones, in particular polyvinylpyrrolidone PVP K30, copovidone polyethylene glycols, propylene glycol, microcrystalline celluloses, sugars and their derivatives, and mixtures thereof.

According to an advantageous embodiment, the binder, when it is not the diluent, is present in the tablet in an amount of 0.01 to 10% by weight with respect to the total weight of the uncoated tablet.

In particular, the lubricant is chosen from the group comprising hydrogenated castor oil, stearic acid, magnesium stearate, calcium stearate, sodium stearyl fumarate, glyceryl palmitostearate, polyoxyethylene stearates. glyceryl behenate, sodium lauryl sulfate, poloxamers, glyceryl monostearate, glyceryl monocaprylocaprate, polyethylene glycol, polyoxyethylene glycol, in particular micronized, leucine, sodium benzoate, talc and mixtures thereof.

According to an advantageous embodiment, the lubricant is the sodium stearyl fumarate. According to an advantageous embodiment, the lubricant is present in the tablet in an amount of 0.01 to 10% by weight, in particular 0.1 to 5%, more particularly 0.2 to 1 or 2%, with respect to the total weight of the uncoated tablet.

The coating agent is in particular chosen from the group comprising polyvinyl alcohols, poly vinyl acetates, vinyl pyrrolidone/acrylates/(lauryl methacrylate) copolymers, acrylates/(C1-2 succinates)/hydroxyacrylates copolymers, polybutyl methacrylates. PVP/(DMAPA acrylates) copolymers, cellulose acetate/phthalate in aqueous dispersion, and cross-linked poly(2-ethylhexyl acrylate).

According to an advantageous embodiment, the coating agent is present in the tablet in an amount of 0.31 to 10% by weight, in particular 0.05 to 8%, more particularly 0.08 to 4 or 6%, with respect to the total weight of the uncoated tablet.

The sweetener is in particular chosen from the group comprising acesulfame potassium, aspartame, cyclamate, mogrosides, saccharin, saccharinates, stevia, sucralose, and polyols, in particular sorbitol, xylitol and lactitol.

According to an advantageous embodiment, the sweetener is present in the tablet in an amount of 0.01 to 10% by weight, in particular 0.05 to 5%, more particularly about 0.1%, with respect to the total weight of the uncoated tablet.

According to an embodiment, the tablet further comprises a disintegrant and/or a slip agent. The disintegrant is in particular chosen from the group comprising calcium or sodium carboxymethyl cellulose, croscarmellose sodium, crospovidone, alginic acid or one of its derivatives, carboxymethyl starch, in particular sodium, pregelatinized starch, and mixtures thereof.

The disintegrant is present in the tablet, for example, in an amount of 0.01 to 10% by weight of the total weight of the uncoated tablet.

The slip agent is in particular chosen from the group comprising colloidal silica, talc, magnesium silicate, calcium stearate, and calcium phosphate.

According to an advantageous embodiment, the slip agent is present in the tablet in an amount of 0.01 to 10% by weight with respect to the total weight of the uncoated tablet.

According to another embodiment, the tablet is free of disintegrant and/or slip agent.

### Definitions

As used in this description, the term “about” refers to a range of values within ±10% of a specific value. For example, the term “about 120 mg” comprises the values of 120 mg±10%, i.e. the values from 108 mg to 132 mg.

For the purposes of this description, percentages refer to percentages by weight of the total weight of the formulation, unless otherwise stated.

As understood here, the value ranges in the form of “x-y” or “from x to y” or “between x and y” include the bounds x and y as well as the integers between these bounds. For example, “1-5”, or “from 1 to 5” or “between 1 and 5” refer to the integers 1, 2, 3, 4 and 5. The preferred embodiments include each individual integer in the value range, as well as any sub-combination of those integers. For example, the preferred values for “1-5” may comprise the integers 1, 2, 3, 4, 5, 1-2, 1-3, 1-4, 1-5, 2-3, 2-4, 2-5, etc.

By “hydroxyurea” (or hydroxycarbamide) is meant the compound of the following formula:

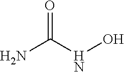

but also all the pharmaceutically acceptable salts of the compound of the above formula.

By “chronic anaemia” is meant a non-acute anaemia, in particular an anaemia of long duration, such as an anaemia lasting more than one month.

By “severe anaemia” is meant an anaemia in which the total haemoglobin level in a patient suffering from said severe anaemia is, before use of hydroxyurea, less than or equal to 7.5 g/dl of blood. This includes in particular an anaemia in which the total haemoglobin level is less than 7 g/dl of blood, or even 6 g/dl of blood, before hydroxyurea use. It may also be an anaemia in which the total haemoglobin level is, before use of hydroxyurea, below 7 g/dl of blood, with poor clinical or functional tolerance.

By “poor clinical tolerance” is meant in particular an impact of the anaemia on the heart (e.g. development or worsening of heart disease, palpitations, systolic murmurs) and/or lungs (e.g. shortness of breath).

By “poor functional tolerance” is meant in particular a difficulty in performing usual activities, particularly due to a lethargy and/or a fatigability.

By “haemoglobin” is meant the iron-containing metalloprotein found in particular in the blood of vertebrates in their red blood cells. Its function is to carry oxygen in the blood.

By “total haemoglobin level” is meant the total amount of haemoglobin in the red blood cells, expressed in grams per liter of blood, and in particular the haemoglobin level measured by one of the methods well known to the person skilled in the art, for example the Drabkin method using colorimetry or the automated hematology methods (which use agents allowing the lysis of the red blood cells and automatic measurement, by photometry).

By “patient suffering from sickle cell disease” is meant in particular the homozygous sickle cell patients (or “SS”, HbS/HbS) and the heterozygous sickle cell patients for which the HbS is in association with an abnormal haemoglobin responsible for a major sickle cell syndrome (S/B thal; S/C, S/D Punjab; S/O-Arab; A/S Antilles; A/S Oman).

By “patient suffering from sickle cell disease and not already undergoing treatment with hydroxyurea” is meant in particular a patient who has never been treated with hydroxyurea, or a patient who was not treated with hydroxyurea at the beginning of the treatment of the severe chronic anaemia, within the meaning of the present invention. In the second case, it is in particular sickle cell patients who have not suffered 3 or more hospitalized vaso-occlusive crisis per year and/or more than one acute chest syndrome, for example in the year preceding the start of the treatment of the severe chronic anaemia within the meaning of the present invention. These are, for example, patients who have had 1 vaso-occlusive crisis in the year preceding the start of the treatment for severe chronic anaemia, or patients who have not had vaso-occlusive crisis. The latter includes, for example, patients who have never had a vaso-occlusive crisis (i.e. patients who have never had a vaso-occlusive crisis in their lives, in particular before the start of the treatment for severe chronic anaemia). These may be patients who do not have vaso-occlusive crisis requiring a hospitalization, or patients who do not have vaso-occlusive crisis, whether hospitalized or not, these crisis being in particular crisis lasting more than 24 or 48 hours. The severe chronic anaemia is in this case “isolated”. In particular, these are patients without vaso-occlusive crisis or acute chest syndrome.

By “vaso-occlusive crisis” is meant in particular a complication of the sickle cell disease characterized by a local obstruction of the blood flow. This vaso-occlusion is usually due to the aggregation of red blood cells in the capillaries. It is usually expressed by acute onset pain, most often at the level of the extremities, of the chest and the back. The occurrence of severe crisis. defined by a duration of more than 24 or 48 hours and requiring the hospitalization to resolve the pain crisis, may require the initiation of a treatment with hydroxyurea.

By “increasing the total haemoglobin level” in the patient is meant an increase in the total haemoglobin level with respect to the total haemoglobin level before use of hydroxyurea, the increase being measured in particular after 1 to 24 months of hydroxyurea use, for example with the hydroxyurea doses defined above.

By “fetal haemoglobin level” is meant the amount of fetal haemoglobin (HbF or α2γ2, the haemoglobin A (HbA), majority in the adults, being of the formula α2β2), and in particular the fetal haemoglobin level measured by HPLC or by the technique of Betké of resistance to the alkaline denaturation (Betké et al, Nature 1959, 184, 1877-4), as for example described by Bardakdjian-Michau et al. (Ann Biol Clin. 2003, 61, 401-9).

As used herein, the term “pharmaceutically acceptable carrier” refers to carriers that are, within the scope of a common sense medical judgment, suitable for the contact with the human and animal tissues without excessive toxicity, irritation, allergic response, or other problematic complications in proportion with a reasonable benefit/risk ratio.

By “film-coated tablet” is meant in particular a tablet comprising an outer film formed from at least one coating agent.

By “dispersible tablet” is meant, in particular, a galenic form for oral administration which disintegrates on contact with a liquid, in particular water, and which disintegrates in particular in water, before administration.

By “rapidly disintegrating tablet” is meant, in particular, a galenic form for oral administration whose disintegration rate is such that, when placed in contact with water, in particular in water. it disintegrates in less than 5 minutes. This allows to provide a suspension that is easy to swallow.

By “diluent” is meant a compound intended to dilute the active substance or the active substances.

By “disintegrant” is meant a compound that contributes to the disintegration of the tablets when administered.

By “binder” is meant a compound allowing the powder particles to be held together.

By “slip agent” is meant a compound which improves the flow of a pulverulent composition. for example before its compression to form a tablet.

By “lubricant” is meant a compound that reduces the frictions, in particular between a tablet and the external environment.

### EXAMPLES

### Example 1: Clinical Trial

This study was conducted with a pharmaceutical composition identical to that of Siklos.

The analysis of the hematological parameters (Hb, HbF . . . ) during the treatment with Siklos was performed in a population of 72 children and adults, with a diagnosis of severe anaemia and who had never received hydroxyurea before this study (HU-naive), and who did not have vaso-occlusive crisis or an acute chest syndrome.

TABLE 1
Demographics of the cohort of sickle cell patients with severe
chronic anaemia, whose total haemoglobin level was less than
or equal to 7.5 g/dl of blood before the study.

| | | Cohort |
|---|---|---|
| Sex | | N = 72 |
| | Male, n (%,) | 32 (44.4%) |
| | Female, n (%) | 40 (55.6%) |
| Age (years) | | N = 72 |
| | Mean ± standard deviation | 16.86 ± 16.49 |
| | Interval | 1.6; 67.6 |
| Age category* | | N = 71 |
| | [2-10 years], n (%) | 36 (50.7%) |
| | [10-16 years], n (%) | 13 (18.3%) |
| | >16 years n (%) | 22 (31.0%) |

*4 patients under 2 years of age are not included in the age categories

The dose of hydroxycarbamide is comprised from 5 to 30 mg/kg/day.

The results of the evaluation of the hematological parameters of the patients are recorded in the table below.

TABLE 2
Measurements taken before the initiation of the treatment with Siklos and
after the initiation at the treatment with Siklos: p-value for the
comparisons between the base value and the value after the initiation
of the treatment with Siklos: § p-test pairwise: 0.001*; $ tests of
the Wilcoxon signed ranks < 0.001*. If applicable: * Value p < 0.05.

| | | Value after | Change with |
|---|---|---|---|
| | | initiation of the | respect to the |
| | Base value | treatment | base value |
| Variation of the haemoglobin | | | |
| (g/dL) 12 months after the | | | |
| initiation of the treatment with | | | |
| Siklos (between 10 and 14 | | | |
| months after the initiation): | | | |
| Number of patients | 41 | 41 | 41 |
| Mean ± standard deviation | 7.16 ± 1.06 | 7.89 ± 1.11 | 0.73 ± 1.30 |
| § p-value | | | 0.001* |
| $ p-value | | | <0.001* |
| Variation of the haemoglobin | | | |
| (g/dL) at least 6 months after | | | |
| the initiation of the treatment | | | |
| with Siklos (between 5 and 14 | | | |
| months after the initiation): | | | |
| Number of patients | 55 | 55 | 55 |
| Mean ± standard deviation | 7.11 ± 0.96 | 7.75 ± 1.16 | 0.65 ± 1.24 |
| § p-value | | | <0.001* |
| $ p-value | | | <0.001* |
| Evolution of the fetal | | | |
| haemoglobin (%) at least | | | |
| 6 months after the initiation | | | |
| of the treatment with Siklos | | | |
| (between 5 and 14 months | | | |
| after the initiation): | | | |
| Number of patients | 23 | 23 | 23 |
| Mean ± standard deviation | 5.78 ± 4.07 | 11.43 ± 7.32 | 5.65 ± 6.51 |
| § p-value | | | <0.001* |
| $ p-value | | | <0.001* |

At 12 months after the initiation of the treatment with hydroxycarbamide, the total of Hb increased from 7.16±1.06 g/dl at the start to 7.89±1.11 g/dl, i.e. a statistically significant increase of 0.73±1.30 (p=0.001). A statistically significant increase in the Hb rate of 0.65±1.24 g/dl (p<0.001) and in the HbF rate (%) of 6.37±6.38 (p<0.001) was also observed after at least 6 months. The mean blood volume increased 12 months after the start of the treatment with hydroxycarbamide (+10.82±11.42, p<0.001) and the absolute number of neutrophil decreased, indicating adequate compliance with the treatment with hydroxycarbamide. The number of reticulocytes decreased, 12 months after the start of the treatment (−91.5±115.6, p<0.001).

### Example 2: Pediatric Clinical Trial

The pediatric clinical trial provided similar results to those obtained in the clinical trial in Example 1.

1. A method of treating severe chronic anaemia in a patient suffering from sickle cell disease and not having vaso-occlusive crisis, the total haemoglobin level in said patient being, before said use of hydroxyurea, less than or equal to 7.5 g/dl of blood, comprising administering to said patient a therapeutically effective amount of hydroxyurea.

2. The method according to claim 1, for increasing the total haemoglobin level in said patient.

3. The method according to claim 1, wherein the total haemoglobin level in the patient, before use of hydroxyurea, is less than 7.4; 7.3; 7.2; 7.1; 7.0; 6.9; 6.8; 6.7; 6.6; 6.5; 6.4; 6.3; 6.2; 6.1 or 6.0 g/dl of blood.

4. The method according to claim 1, wherein the fetal haemoglobin level is between 5 and 25% of the total haemoglobin level in the patient, before use of hydroxyurea.

5. The method according to claim 1, wherein the patient is aged, before use of hydroxyurea, of 9 months or more.

6. The method according to claim 1, wherein the total haemoglobin level in the patient increases by more than 9%.

7. The method according to claim 1, wherein the total haemoglobin level in the patient increases to more than 8.0 g/dl of blood.

8. The method according to claim 1, wherein the pharmaceutical composition comprises hydroxyurea in an amount of from 50 to 1000 mg.

9. The method according to claim 1, wherein the pharmaceutical composition is a tablet, or a liquid pharmaceutical form such as a syrup or a drinkable solution.
